(19) United States
(12) Patent Application Publication
(10) Pub. No.: US 2013/0197126 A1
(43) Pub. Date: Aug. 1, 2013

(54) Paste-Like Bone Cement

(71) Applicant: Heraeus Medical GmbH, Wehrheim (DE)

(72) Inventor: Sebastian Vogt, Erfurt (DE)

(73) Assignee: Heraeus Medical GmbH, Wehrheim (DE)

(21) Appl. No.: 13/753,231

(22) Filed: Jan. 29, 2013

(30) Foreign Application Priority Data
Jan. 30, 2012 (DE) 102012001636.0

(51) Int. Cl. A61K 9/10

(52) U.S. Cl. CPC A61K 9/10
USPC 523/116

(57) ABSTRACT

The present invention relates to a paste, a kit, and the use of the paste for mechanical fixation of articular endoprostheses. The paste contains at least one polymer for radical polymerisation, at least one polymer that is soluble in at least one monomer for radical polymerisation, and at least one filling agent that is poorly soluble or insoluble in at least one monomer for radical polymerisation, whereby the filling agent is a particulate cross-linked polymethacrylate produceable through polymerisation of methacrylic acid esters, whereby

i) at least 15% by weight of the methacrylic acid esters used for polymerisation are multi-functional methacrylic acid esters, relative to the total weight of the methacrylic acid esters used in the polymerisation, and

ii) at least 90% by weight of the particles of the filling agent, relative to the total weight of the filling agent, have a particle size of no more than 74 μm.

The present invention relates to a paste, a kit, the use of a filling agent that is poorly soluble or insoluble in a monomer for radical polymerisation, and the use of a paste or of a paste produced from a kit for mechanical fixation of articular endoprostheses, for covering skull defects, for filling bone cavities, for femuroplasty, for vertebroplasty, for kyphoplasty, for the manufacture of spacers or for the production of carrier materials for local antibiotics therapy.

Conventional polymethylmethacrylate bone cements (PMMA bone cements) have been known for decades and are based on the ground-breaking work of Sir Charnley (Charnley, J.: “Anchor-age of the femoral head prosthesis of the shaft of the femur”; J. Bone Joint Surg. 42 (1960) 28-30). The basic structure of PMMA bone cements has remained the same ever since. PMMA bone cements consist of a liquid monomer component and a powder component. The monomer component generally contains (i) the monomer, methylmethacrylate, and (ii) an activator (e.g. N,N-dimethyl-p-toluidine) dissolved therein. The powder component comprises (i) one or more polymers that are made by polymerisation, preferably by suspension polymerisation, based on methylmethacrylate and co-monomers, such as styrene, methylacrylate or similar monomers, (ii) a radio-opaquer, and (iii) an initiator, (e.g. dibenzoylperoxide). Mixing the powder component and the monomer component, the polymers of the powder component in the methylmethacrylate swell which generates a dough that can be shaped plastically. Simultaneously, the activator, N,N-dimethyl-p-toluidine, reacts with dibenzoylperoxide which disintegrates and forms radicals in the process. The radicals thus formed trigger the radical polymerisation of the methylmethacrylate. Upon advancing polymerisation of the methylmethacrylate, the viscosity of the cement dough increases until the cement dough solidifies and thus is cured.

Paste-like polymethylmethacrylate bone cements containing a methacrylate monomer for radical polymerisation, a polymer that is soluble in said methacrylate monomer, and a particulate polymer that is insoluble in said methacrylate monomer have been described as an alternative to the conventional powder-liquid polymethylmethacrylate bone cements in unexamined German patent applications DE-A-10 2007 052 116, DE-A-10 2007 050 762, and DE--A-10 2007 050 763. Paste-like polymethylmethacrylate bone cements of this type can be present as one-component systems (in this case, the paste contains all components required for curing, in particular an activatable radical initiator, e.g. a photoinitiator or a photoinitiator system) or as two-component systems (in this case, the system comprises two pre-mixed pastes that are stabile on storage and one of which comprises a radical polymerisation initiator and the other comprises a polymerisation activator). Referring to two-component systems, a distinction is made between a “symmetrical system” (in this case both pastes contain a particulate polymer that is insoluble in the methacrylate monomer) and “non-symmetrical systems” (in this case, only one of the two pastes contains a particulate polymer that is insoluble in the methacrylate monomer).

As a result of the selected composition, the bone cement produced from the pastes described above possesses sufficiently high viscosity and cohesion in order to withstand the pressure from bleeding until it is fully cured. Owing to the advancing polymerisation, the paste is cured while the methacrylate monomers are consumed.

Inorganic filling agents that are insoluble in the methacrylate monomer lead to pronounced brittleness of the cured paste cements which often leads to fracturing in the grains. In turn, the use of organic particulate filling agents that are swellable in methacrylate monomers showed that the cured cement, after pronounced uptake of the methacrylate monomers, attains its final hardness not right after the cement pastes cure, but only after a delay. In all known polymethylmethacrylate bone cements based on cement powder and monomer liquid, the freezing of the polymerisation invariably leads to a certain residual monomer content which after-polymerises only over the course of hours to days. More pronounced after-curing was observed during the curing of paste-like bone cements though. This is caused since the methacrylate monomer that is taken up through swelling into the swelled polymer particles participates only incompletely in the polymerisation while the cement matrix surrounding the swelled particles is cured. This means that non-polymerised methacrylate monomer can diffuse from the particles after the cement matrix is cured and temporarily act as a plasticiser until it is after-polymerised as well. On the other hand, tests have shown that organic filling agents taking up no or minimal amounts of methacrylate monomer only are bonded to the cement matrix quite poorly and thus mechanically weaken the cured cement.

The present invention was based on the object to overcome the disadvantages of prior art bone cement systems that are based on pastes, in particular with regard to the one-component systems and two-component systems described above.

The present invention was also based on the object to provide a bone cement paste that shows little post-cure tendency and concurrently affords a bone cement after curing that has advantageous mechanical stability, in particular low brittleness.

Said object was met through the use of a particulate cross-linked polymethacrylate that takes up only small amounts of methacrylate monomer through swelling and still forms a good bond to the paste matrix that is obtained during the curing of the bone cement through polymerisation of the methacrylate monomer in the presence of a polymer that is soluble in the methacrylate monomer, in conventional one-component or two-component systems as a particulate filling agent that is insoluble in a methacrylate monomer.

A contribution to meeting the objects stated above is made by a paste containing at least one polymer for radical polymerisation, at least one polymer that is soluble in said at least one monomer for radical polymerisation, and at least one filling agent that is poorly soluble or insoluble in said at least one monomer for radical polymerisation, whereby the filling agent is a particulate crosslinked polymethacrylate that can be produced, preferably was produced, through polymerisation of methacrylic acid esters, whereby

i) at least 15% by weight of the methacrylic acid esters used for polymerisation are multifunctional methacrylic acid esters, relative to the total weight of the methacrylic acid esters used in the polymerisation (this refers to the polymerisation leading to the production of the particulate cross-linked polymethacrylates rather than the polymerisation proceeding while the paste cures), and

ii) at least 90% by weight of the particles of the filling agent, relative to the total weight of the filling agent, have a particle size of no more than 74 μm.

As a matter of principle, the paste according to the invention can be a one-component system of the type described above or can be obtained through mixing the two pastes of a two-component system of the type described above.

The paste according to the invention contains, as a component, at least one monomer for radical polymerisation, whereby this is preferably a methacrylate monomer, in particular a methacrylate monomer that is liquid at a temperature of 25° C. and a pressure of 1,013 hPa.

Preferably, the methacrylate monomer is a methacrylic acid ester. Preferably, the methacrylic acid ester is a mono-functional methacrylic acid ester. Preferably, said substance is hydrophobic. The use of hydrophobic monofunctional methacrylic acid esters allows later increases in bone cement volume due to the uptake of water and thus damage to the bone to be prevented. According to a preferred embodiment, the monofunctional methacrylic acid ester is hydrophobic if it contains no further polar groups aside from the ester group. The monofunctional hydrophobic methacrylic acid ester preferably comprises no carboxyl groups, hydroxyl groups, amide groups, sulfonic acid groups, sulfate groups, phosphate groups or phosphonate groups.

The esters preferably are alkyl esters. According to the invention, cycloalkyl esters are also included in alkyl esters. According to a preferred embodiment, the alkyl esters are esters of methacrylic acid and alcohols comprising 1 to 20 carbon atoms, more preferably 1 to 10 carbon atoms, even more preferably 1 to 6 carbon atoms, and particularly preferably 1 to 4 carbon atoms. The alcohols can be substituted or non-substituted and preferably are non-substituted. Moreover, the alcohols can be saturated or unsaturated and preferably are saturated.

The monomer for radical polymerisation used according to the invention preferably has a molar mass of less than 1,000 g/mol. This also comprises monomers for radical polymerisation that are components of a mixture of monomers, whereby at least one of the monomers for radical polymerisation of the mixture of monomers has a defined structure with a molar mass of less than 1,000 g/mol.

The monomer for radical polymerisation is preferably characterised in that an aqueous solution of the monomer for radical polymerisation has a pH in the range of 5 to 9, preferably in the range of 5.5 to 8.5, even more preferably in the range of 6 to 8, and particularly preferably in the range of 6.5 to 7.5.

According to a particularly preferred embodiment, the methacrylate monomer is a methacrylic acid methylester, methacrylic acid ethylester or a mixture of said two monomers.

Preferably, the paste according to the invention contains an amount of the monomer for radical polymerisation in a range of 15 to 85% by weight, more preferably 20 to 70% by weight, even more preferably 25 to 60% by weight, and particularly preferably 25 to 50% by weight, each relative to the total weight of the paste according to the invention.

The paste according to the invention contains, as further component, at least one polymer that is soluble in said at least one monomer for radical polymerisation. According to the invention, a polymer is soluble in the polymerisable monomer, if at least 10 g/l, preferably at least 25 g/l, more preferably at least 50 g/l, and particularly preferably at least 100 g/l of the polymer dissolve in said polymerisable monomer. The polymer that is soluble in the polymerisable monomer can be a homopolymer or a copolymer. Said soluble polymer preferably is a polymer with a mean (by weight) molar mass of at least 150,000 g/mol. The soluble polymer can, for example, be a polymer or copolymer of a methacrylic acid ester. According to a particularly preferred embodiment, the at least one soluble polymer is selected from the group consisting of polymethacrylic acid methylester (PMMA), polymethacrylic acid ethylester (PMAE), polymethacrylic acid propylester (PMAP), polymethacrylic acid isopropylester, poly(methylmethacrylate-comethylacrylate), poly(styrene-co-methylmethacrylate), and a mixture of at least two of said polymers.

The amount of the polymer that is soluble in said monomer for radical polymerisation that is present in the paste usually is in a range of 1 to 85% by weight, relative to the total weight of the paste according to the invention.

Moreover, the paste according to the invention contains at least one filling agent that is poorly soluble or insoluble in the at least one monomer for radical polymerisation, whereby the filling agent is a particulate cross-linked polymethacrylate that was produced through polymerisation of methacrylic acid esters, whereby

i) at least 15% by weight of the methacrylic acid esters used for polymerisation are multifunctional methacrylic acid esters, relative to the total weight of the methacrylic acid esters used in the polymerisation, and

ii) at least 90% by weight of the particles of the filling agent, relative to the total weight of the filling agent, have a particle size of no more than 74 μm.

The poorly soluble or insoluble filling agent is a solid substance at room temperature that is capable of increasing the viscosity of the mixture made up of the other components contained in the paste according to the invention. The filling agent should be biocompatible.

The filling agent is poorly soluble or insoluble in the monomer for radical polymerisation. According to the invention, the filling agent is poorly soluble or insoluble in the monomer for radical polymerisation, if the solubility of the filling agent in the monomer for radical polymerisation at a temperature of 25° C. is less than 50 g/l, preferably less than 25 g/l, more preferably less than 10 g/l, even more preferably less than 5 g/l, and yet more preferably less than 0.5 g/l, whereby it is most preferred that the poorly soluble or insoluble filling agent does not dissolve at all or only in negligible amounts in the monomer for radical polymerisation.

Moreover, the filling agent is particulate, whereby at least 90% by weight, particularly preferably at least 95% by weight, and most preferably 100% by weight of the particles of the filling agent, each relative to the total weight of the filling agent, have a particle size of no more than 74 μm, particularly preferably of no more than 63 μm, and most preferably of no more than 53 μm. The phrase, “whereby at least 90% by weight of the particles of the filling agent, relative to the total weight of the filling agent, have a particle size of no more than 74 μm”, is meant to express that maximally 10% by weight of the particles are retained on a sieve when the filling agent is screened through a sieve with a mesh of 74 μm (mesh-200 sieve). Likewise, the phrase, “whereby at least 90% by weight of the particles of the filling agent, relative to the total weight of the filling agent, have a particle size of no more than 63 μm”, is meant to express that maximally 10% by weight of the particles are retained on a sieve when the filling agent is screened through a sieve with a mesh of 63 μm (mesh-230 sieve) and the phrase, “whereby at least 90% by weight of the particles of the filling agent, relative to the total weight of the filling agent, have a particle size of no more than 53 μm”, is meant to express that maximally 10% by weight of the particles are retained on a sieve when the filling agent is screened through a sieve with a mesh of 53 μm (mesh-270 sieve).

The particulate filling agent is a cross-linked polymethacrylate that can be produced, preferably was produced, through polymerisation of methacrylic acid esters, whereby at least 15% by weight, particularly preferably at least 20% by weight of the methacrylic acid esters used for polymerisation are multifunctional methacrylic acid esters, each relative to the total weight of the methacrylic acid esters used in the polymerisation. Preferably, the cross-linked polymethylmethacrylate has a mean (by weight) molar mass of at least 150,000 g/mol. The specification of the molar mass refers to the mean (by weight) molar mass determined by gel permeation chromatography (GPC).

According to a particular refinement of the paste according to the invention, said paste contains two different particulate cross-linked polymethacrylates P1 and P2 that implement features i) and ii), whereby
P1 was produced through polymerisation of 90 to 100% by weight multifunctional methacrylic acid esters and 0 to 10% by weight monofunctional methacrylic acid esters, each relative to the total weight of the methacrylic acid esters used in the polymerisation, and
P2 was produced through polymerisation of 20 to 40% by weight multifunctional methacrylic acid esters and 60 to 80% by weight monofunctional methacrylic acid esters, each relative to the total weight of the methacrylic acid esters used in the polymerisation.

The multifunctional methacrylic acid ester preferably is a multifunctional methacrylic acid ester selected from the group consisting of ethylene glycol dimethacrylate, butan-1,4-diol-dimethacrylate, hexan-1,6-diol-dimethacrylate, dodecan-1,12-diol-dimethacrylate, diethylene glycol dimethacrylate, trimethylene glycol trimethacrylate, glycerol-1,2,3-trimethacrylate, and pentaerythritoltetramethacrylate. In as far as other co-monomers are used for producing the particulate cross-linked polymethacrylates, said co-monomers preferably are monofunctional methacrylic acid esters, particularly preferably methylmethacrylate.

The particulate cross-linked polymethacrylate can be spherical or aspherical in shape. Approximately spherical cross-linked polymethylmethacrylate is produced through suspension polymerisation. The particulate cross-linked polymethacrylate of aspherical shape is produced through thermally induced bulk polymerisation of methacrylic acid esters and disintegrated to the desired grain size by grinding the polymer after polymerisation. The disintegrated polymer is called chipped polymer.

If applicable, the paste according to the invention can contain, aside from the particulate crosslinked polymethacrylate described above, further filling agents, such as inorganic salts, inorganic oxides, metals or metal alloys.

Preferably, the inorganic salt is a salt of an element selected from the second main group of the periodic system of elements. According to a preferred embodiment, the inorganic salt is a calcium, strontium or barium salt. According to a particularly preferred embodiment, the inorganic salt is calcium sulfate, barium sulfate or calcium carbonate. The inorganic oxide that can be used as filling agent can preferably be a metal oxide. According to a preferred embodiment, the inorganic oxide is a transition metal oxide. According to a particularly preferred embodiment, the inorganic oxide is titanium dioxide or zirconium dioxide. The metal that can be used as filling agent can, for example, be a transition metal. According to a preferred embodiment, the metal is tantalum or tungsten. The metal alloy that can be used as filling agent is an alloy of at least two metals. Preferably, the alloy contains at least one transition metal. According to a particularly preferred embodiment, the alloy comprises at least tantalum or tungsten. The alloy can also be an alloy of tantalum and tungsten.

The amount of filling agent, particularly preferably the amount of particulate cross-linked polymethacrylate, in the paste according to the invention, preferably is 15 to 85% by weight, particularly preferably 15 to 80% by weight, and even more preferably 20 to 75% by weight, each relative to the total weight of the paste according to the invention.

Moreover, the paste according to the invention can contain at least one polymerisation initiator, at least one polymerisation accelerator or at least one polymerisation initiator and one polymerisation accelerator.

In the case of a one-component system, the polymerisation initiator preferably is an activatable polymerisation initiator, e.g. a photoinitiator that is dissolved or suspended in the paste or a photoinitiator system that is dissolved or suspended in the paste. It is feasible just as well to provide an initiator or initiators where it/they are temporarily in contact with the paste, for example in a container part, a dosing facility or a transport cannula. Moreover, in a one-component system, the paste according to the invention can also contain an electrically conductive radio-opaquer aside from the activatable polymerisation initiator. Particles made of cobalt, iron, NdFeB, SmCo, cobalt-chromium steel, zirconium, hafnium, titanium, titanium-aluminium-silicon alloys, and titanium-niobium alloys having a particle size of 0.5-500 μm are particularly well-suited in this context. It is feasible to induce eddy currents in said electrically conductive radio-opaquer through alternating magnetic fields with a frequency in the range of 500 Hz to 50 kHz which cause the radio-opaquer to heat up. Due to heat transmission, the initiator is heated as well and induced to thermally disintegrate.

In the case of a paste according to the invention that was obtained through combining two pastes of a two-component system, said paste preferably contains at least one polymerisation initiator (that was contained in the one paste of the two-component system) and at least one polymerisation accelerator (that was contained in the other paste of the two-component system).

Conceivable as polymerisation initiator are, in particular, peroxides and barbituric acid derivatives, whereby preferably at least 1 g/l, more preferably at least 3 g/l, even more preferably at least 5 g/l, and particularly preferably at least 10 g/l of the peroxides and barbituric acid derivatives can dissolve(s) in the polymerisable monomer at a temperature of 25° C.

According to the invention, a peroxide is understood to mean compounds that contain at least one peroxo group (—O—O—). The peroxide preferably comprises no free acid groups. The peroxide can be an inorganic peroxide, such as, for example, a toxicologically acceptable hydroperoxide, or an organic peroxide. According to a particularly preferred embodiment, the peroxide is selected from the group consisting of dibenzoyl peroxide and dilauroyl peroxide.

The barbituric acid derivative preferably is a barbituric acid derivative selected from the group consisting of 1-mono-substituted barbiturates, 5-mono-substituted barbiturates, 1,5-di-substituted barbiturates, and 1,3,5-tri-substituted barbiturates. According to a particular refinement of the paste according to the invention, the barbituric acid derivative is selected from the group consisting of 1,5-di-substituted barbiturates and 1,3,5-tri-substituted barbiturates.

There is no limitation with regard to the type of substituents on the barbituric acid. The substituents can, for example, be aliphatic or aromatic substituents. In this context, alkyl, cycloalkyl, allyl or aryl substituents can be preferred. The substituents can also include hetero atoms. In particular, the substituents can be thiol substituents. Accordingly, 1,5-disubstituted thiobarbiturates or 1,3,5-trisubstituted thiobarbiturates can be preferred. According to a preferred embodiment, the substituents each have a length of 1 to 10 carbon atoms, more preferably a length of 1 to 8 carbon atoms, and particularly preferably a length in the range of 2 to 7 carbon atoms. According to the invention, barbiturates bearing one substituent each at position 1 and position 5 or a substituent at positions 1, 3, and 5 are preferred. According to another preferred embodiment, the barbituric acid derivative is a 1,5-disubstituted barbiturate or a 1,3,5-trisubstituted barbiturate. According to a particularly preferred embodiment, the barbituric acid derivative is selected from the group consisting of 1-cyclohexyl-5-ethyl-barbituric acid, 1-phenyl-5-ethyl-barbituric acid, and 1,3,5-trimethyl-barbituric acid.

Heavy metal compound selected from the group consisting of heavy metal salts and heavy metal complexes, are preferred as polymerisation accelerator, whereby it has proven to be particularly advantageous for the heavy metal compound to be poorly soluble, preferably even insoluble, in the monomer for radical polymerisation. A heavy metal compound is considered to be poorly soluble or insoluble in the monomer for radical polymerisation if less than 1 g/l, preferably less than 0.1 g/l, even more preferably less than 0.01 g/l, yet more preferably less than 0.001 g/l, even yet more preferably less than 0.0001 g/l, and most preferably no significant amounts of the heavy metal compound at all can dissolve in the monomer for radical polymerisation at a temperature of 25° C. (i.e. the heavy metal compound is insoluble in the monomer for radical polymerisation).

According to the invention, heavy metal compounds shall be understood to mean metals with a density of at least 3.5, preferably of at least 5, at a temperature of 20° C. According to a preferred embodiment, the heavy metal compound is a basic heavy metal compound. Basic heavy metal compound shall be understood to mean a heavy metal compound which, when dissolved or suspended in water, has a pH of at least 7.0, preferably at least 8, and even more preferably at least 8.5. According to a particularly preferred embodiment, the heavy metal compounds are compounds of metals that can change their oxidation state. Copper (II), iron (II), iron (III), manganese (II), manganese (III), cobalt (II), and cobalt (III) compounds are preferred according to the invention in this context with copper(II) compounds being particularly preferred. Provided they are heavy metal compounds that are poorly soluble or insoluble in the monomer for radical polymerisation, the heavy metal compounds according to the invention are preferably capable, in the presence of the barbituric acid derivatives, of converting into a form that is soluble in the monomer for radical polymerisation. According to the invention, the heavy metal compounds preferably are heavy metal salts or heavy metal complexes. The heavy metal salts preferably are halides, hydroxides, carbonates or carbonic acid salts of heavy metals. Copper (II), iron (II), iron (III), manganese (II), manganese (III), cobalt (II), and cobalt (III) salts are preferred heavy metals salts. Moreover, halide salts are conceivable as heavy metal compound. The halide salt can preferably be selected from the group consisting of heavy metal chlorides and bromides. According to a particular embodiment, the halide salt is a compound selected from the group consisting of manganese(II) chloride, iron(II) chloride, iron(III) chloride, cobalt(II) chloride, and cobalt(III) chloride. According to a particularly preferred embodiment, the heavy metal salt is selected from the group consisting of copper(II) hydroxide, basic copper(II) carbonate or a mixture of at least two thereof, in particular a mixture of copper(II) hydroxide and basic copper(II) carbonate.

The paste according to the invention can contain a (total) amount of up to 5% by weight, relative to the total weight of the paste according to the invention, of the polymerisation initiator, polymerisation accelerator or polymerisation initiator and polymerisation accelerator.

The paste according to the invention can contain further ingredients aside from the components specified above.

According to a preferred embodiment of the paste according to the invention, said paste can contain at least one radio-opaquer. The radio-opaquer can be a common radio-opaquer in this field. Suitable radio-opaquers can be soluble or insoluble in the monomer for radical polymerisation. The radio-opaquer is preferably selected from the group consisting of metal oxides (such as, for example, zirconium oxide), barium sulfate, toxicologically acceptable heavy metal particles (such as, for example, tantalum), ferrite, magnetite (supramagnetic magnetite also, if applicable), and biocompatible calcium salts. Said radio-opaquers preferably have a mean particle diameter in the range of 10 nm to 500 μm. Moreover, conceivable radio-opaquers also include esters of 3,5-bis(acetamido)-2,4,6-triiodobenzoic acid, gadolinium compounds, such as gadolinium chelate involving the esters of 1,4,7,10-tetraazacyclododecan-1,4,7,10-tetraacetic acid (DOTA).

According to a further preferred embodiment, the paste according to the invention can contain at least one colourant. The colourant can be a common colourant in this field and preferably can be a food colourant. Moreover, the colourant can be soluble or insoluble in the at least one monomer for radical polymerisation. According to a particularly preferred embodiment, the colourant is selected from the group consisting of E101, E104, E132, E141 (chlorophyllin), E142, riboflavin, and lissamine green. According to the invention, the term, colourant, shall also include colour varnishes, such as, for example, colour varnish green, the aluminium salt of a mixture of E104 and E132.

According to a further preferred embodiment, the paste according to the invention can contain at least one pharmaceutical agent. The at least one pharmaceutical agent can be present in the paste according to the invention in dissolved or suspended form. The pharmaceutical agent can preferably be selected from the group consisting of antibiotics, antiphlogistic agents, steroids, hormones, growth factors, bisphosphonates, cytostatic agents, and gene vectors. According to a particularly preferred embodiment, the at least one pharmaceutical agent is an antibiotic. Preferably, the at least one antibiotic is selected from the group consisting of aminoglyoside antibiotics, glycopeptide antibiotics, lincosamide antibiotics, gyrase inhibitors, carbapenems, cyclic lipopeptides, glycylcyclines, oxazolidones, and polypeptide antibiotics. According to a particularly preferred embodiment, the at least one antibiotic is a member selected from the group consisting of gentamicin, tobramycin, amikacin, vancomycin, teicoplanin, dalbavancin, lincosamine, clindamycin, moxifloxacin, levofloxacin, ofloxacin, ciprofloxacin, doripenem, meropenem, tigecycline, linezolide, eperezolide, ramoplanin, metronidazole, tinidazole, omidazole, and colistin, as well as salts and esters thereof. Accordingly, the at least one antibiotic can be selected from the group consisting of gentamicin sulfate, gentamicin hydrochloride, amikacin sulfate, amikacin hydrochloride, tobramycin sulfate, tobramycin hydrochloride, clindamycin hydrochloride, lincosamine hydrochloride, and moxifloxacin. The at least one antiphlogistic agent is preferably selected from the group consisting of non-steroidal antiphlogistic agents and glucocorticoids. According to a particularly preferred embodiment, the at least one antiphlogistic agent is selected from the group consisting of acetylsalicylic acid, ibuprofen, diclofenac, ketoprofen, dexamethasone, prednisone, hydrocortisone, hydrocortisone acetate, and fluticasone. The at least one hormone is preferably selected from the group consisting of serotonin, somatotropin, testosterone, and estrogen. Preferably, the at least one growth factor is selected from the group consisting of Fibroblast Growth Factor (FGF), Transforming Growth Factor (TGF), Platelet Derived Growth Factor (PDGF), Epidermal Growth Factor (EGF), Vascular Endothelial Growth Factor (VEGF), insulin-like growth factors (IGF), Hepatocyte Growth Factor (HGF), Bone Morphogenetic Protein (BMP), interleukin-1B, interleukin 8, and nerve growth factor. The at least one cytostatic agent is preferably selected from the group consisting of alkylating agents, platinum analogues, intercalating agents, mitosis inhibitors, taxanes, topoisomerase inhibitors, and antimetabolites. The at least one bisphosphonate is preferably selected from the group consisting of zoledronate and aledronate.

According to a further preferred embodiment, the paste according to the invention can contain at least one biocompatible elastomer. Preferably, the biocompatible elastomer is particulate. Preferably, the biocompatible elastomer is soluble in the at least one monomer for radical polymerisation. The use of butadiene as biocompatible elastomer has proven to be particularly well-suited.

According to a further preferred embodiment, the paste according to the invention can contain at least one monomer having adsorption groups. An amide group, for example, can be an adsorption group. Accordingly, the monomer with adsorption group can, for example, be methacrylic acid amide. Using at least one monomer with adsorption groups would allow the binding of the bone cement to articular endoprostheses to be influenced in a targeted manner.

According to a further preferred embodiment, the paste according to the invention can contain at least one stabiliser. The stabiliser should be suitable to prevent spontaneous polymerisation of the monomers for radical polymerisation that are contained in the paste. Moreover, the stabiliser should not undergo interfering interactions with the other ingredients contained in the paste according to the invention. Stabilisers of said type are known according to the prior art. According to a preferred embodiment, the stabiliser is 2,6-di-tert-butyl-4-methylphenol and/or 2,6-di-tert-butyl-phenol.

A kit comprising a paste A and a paste B also makes a contribution to a solution meeting the object specified above,

whereby

(a) paste A contains
(a1) at least one monomer for radical polymerisation;
(a2) at least one polymer that is soluble in (a1); and
(a3) at least one polymerisation initiator;

(b) paste B contains
(b1) at least one monomer for radical polymerisation;
(b2) at least one polymer that is soluble in (b1); and
(b3) at least one polymerisation accelerator;

and whereby at least one of the pastes A and B contains, as component (a4) or (b4), respectively, at least one filling agent that is poorly soluble or insoluble in (a1) or (b1), respectively, whereby the filling agent is a particulate cross-linked polymethacrylate that can be produced, preferably was produced, through polymerisation of methacrylic acid esters, whereby

i) at least 15% by weight of the methacrylic acid esters used for polymerisation are multifunctional methacrylic acid esters, relative to the total weight of the methacrylic acid esters used in the polymerisation, and

ii) at least 90% by weight of the particles of the filling agent, relative to the total weight of the filling agent, have a particle size of no more than 74 μm.

According to the invention, a kit shall be understood to be a system made up of at least two components. Although reference to two components (i.e. paste A and paste B) is made in the following, the kit can just as well contain more than two components, for example three, four, five or more than five components, according to need. The individual components preferably are provided to be packaged separate from each other such that the ingredients of the one kit component do not contact the ingredients of another kit component. Accordingly, it is feasible, for example, to package the respective kit components separate from each other and to store them together in a reservoir container.

The components described above, in the context of the paste according to the invention, as preferred monomer for radical polymerisation, as polymer that is soluble in said monomer, as polymerisation initiator, as polymerisation accelerator, and as particulate cross-linked polymethacrylate are preferred as monomer (a1) and/or (b1) for radical polymerisation, as polymer that is soluble in said monomer (a1) and/or (b1), as polymerisation initiator (a3), as polymerisation accelerator (b3), and as particulate cross-linked polymethacrylate (a4) and/or (b4).

Preferably, paste A and paste B contain an amount of the at least one monomer for radical polymerisation (a1) and/or (b1) in a range of 15 to 85% by weight, more preferably 20 to 70% by weight, even more preferably 25 to 60% by weight, and particularly preferably 25 to 50% by weight, each relative to the total weight of paste A and/or paste B.

Preferably, paste A contains an amount of the polymerisation initiator (a3) in a range of 0.1 to 10% by weight, more preferably in a range of 0.5 to 8% by weight, and even more preferably in a range of 1 to 5% by weight, each relative to the total weight of paste A.

Preferably, paste B contains an amount of the polymerisation accelerator (b3) in a range of 0.0005 to 0.5% by weight, more preferably in a range of 0.001 to 0.05% by weight, and particularly preferably in a range of 0.001 to 0.01% by weight, each relative to the total weight of paste B.

Provided one of the pastes of the kit according to the invention contains the poorly soluble or insoluble filling agent and the other paste contains no poorly soluble or insoluble filling agent at all or contains a negligible amount of poorly soluble or insoluble filling agent as compared to the amount present in the other paste, the kit is called “asymmetrical”. In contrast, a so-called “symmetrical” kit has approximately comparable amounts of the poorly soluble or insoluble filling agent present in both pastes.

Moreover, paste B can contain, as further component of the initiator system, at least one alkali or alkaline earth halide (b5) of the type described in DE 10 2010 024 653 A1, whereby it has proven to be advantageous for said alkali or alkaline earth halide (b5) to be soluble in the monomer for radical polymerisation (b1).

In principle, F⁻, Cl⁻, and Br⁻ are conceivable as halide anion with Cl⁻ being particularly preferred. Particularly preferred alkali or alkaline earth halides include potassium chloride, sodium chloride, calcium chloride, and magnesium chloride with lithium chloride being most preferred as alkali chloride (b5).

Moreover, it is also preferred in this context that paste B contains an amount of the at least one alkali or alkaline earth halide (b5) in a range of 0.001 to 7.5% by weight, more preferably in a range of 0.01 to 5% by weight, even more preferably in a range of 0.1 to 2.5% by weight, and most preferably in a range of 0.5 to 1.5% by weight, each relative to the total weight of paste B.

Moreover, pastes A and/or B can contain further additives aside from the components described above, such as radio-opaquers, colourants, pharmaceutical agents, biocompatible elastomers, monomers having adhesion groups or stabilisers, whereby the components described above, in the context of the paste according to the invention, as preferred radio-opaquers, colourants, pharmaceutical agents, biocompatible elastomers, monomers having adhesion groups, and stabilisers are preferred here as well.

According to a first particular refinement of the kit according to the invention, the kit is an “asymmetrical” kit. It is preferred in this context that paste A contains 20 to 70% by weight, particularly preferably 25 to 60% by weight, even more preferably 30 to 55% by weight, and most preferably 34 to 47% by weight, each relative to the total weight of paste A, of the filling agent (a4) that is insoluble in (a1), and paste B contains less than 5% by weight, particularly preferably less than 1% by weight, even more preferably less than 0.1% by weight, and yet more preferably less than 0.01% by weight, each relative to the total weight of paste B, of the filling agent (b4) that is insoluble in (b1), whereby it is most preferred that paste B contains no filling agent (b4) that is insoluble in (b1) at all.

Moreover, in the context of said first particular refinement of the kit according to the invention, it is preferred that paste A contains an amount of a polymer (a2) that is soluble in (a1) in a range of 1 to 25% by weight, particularly preferably in a range of 2 to 20% by weight, even more preferably in a range of 2 to 18% by weight, and most preferably in a range of 3 to 16% by weight, each relative to the total weight of paste A, and paste B contains an amount of a polymer (b2) that is soluble in (b1) in a range of 25 to 85% by weight, particularly preferably in a range of 35 to 85% by weight, even more preferably in a range of 40 to 80% by weight, and most preferably in a range of 50 to 75% by weight, each relative to the total weight of paste B.

Moreover, it is preferred in the context of said first particular refinement of the kit according to the invention that the weight ratio of filling agent (b4) that is insoluble in (b1) to the at least one polymer (b2) that is soluble in (b1) is no more than 0.2, more preferably no more than 0.15, even more preferably no more than 0.1, yet more preferably no more than 0.05, particularly preferably no more than 0.02, and even more particularly preferably is equal to 0.

According to a second particular refinement of the kit according to the invention, the kit is a “symmetrical” kit. It is preferred in this context that paste A contains 15 to 85% by weight, particularly preferably 15 to 80% by weight, and even more preferably 20 to 75% by weight, each relative to the total weight of paste A, of the filling agent (a4) that is insoluble in (a1), and paste B contains 15 to 85% by weight, particularly preferably 15 to 80% by weight, and even more preferably 20 to 75% by weight, each relative to the total weight of paste B, of the filling agent (b4) that is insoluble in (b1).

Moreover, in the context of said second particular refinement of the kit according to the invention, it is preferred that paste A contains an amount of a polymer (a2) that is soluble in (a1) in a range of 5 to 50% by weight, particularly preferably in a range of 10 to 40% by weight, and even more preferably in a range of 20 to 30% by weight, each relative to the total weight of paste A, and/or paste B contains an amount of a polymer (b2) that is soluble in (b1) in a range of 5 to 50% by weight, particularly preferably in a range of 10 to 40% by weight, and even more preferably in a range of 20 to 30% by weight, each relative to the total weight of paste B.

According to the invention, the purpose of the paste and/or kit according to the invention containing at least pastes A and B is the production of bone cement.

Referring to the kit, for this purpose, the at least two pastes A and B are mixed with each other, upon which another paste, paste C, is obtained. The mixing ratio preferably is 0.5 to 1.5 parts by weight of paste A and 0.5 to 1.5 parts by weight of paste B. According to a particularly preferred embodiment, the fraction of paste A is 30 to 70% by weight and the fraction of paste B is 30 to 70% by weight, each relative to the total weight of pastes A and B, respectively. Mixing can be effected with common mixing devices, for example a static mixer or a dynamic mixer.

Paste C that is ultimately obtained after mixing the pastes of the kit is tack-free according to the ISO 5833 standard and can be processed without delay.

The bone cement generated from paste C by curing attains high strength approximately six to eight minutes after mixing the pastes contained in the kit.

According to a preferred embodiment, paste C and/or the kit according to the invention can be used for mechanical fixation of articular endoprostheses, for covering skull defects, for filling bone cavities, for femuroplasty, for vertebroplasty, for kyphoplasty, for the manufacture of spacers, and for the production of carrier materials for local antibiotics therapy.

In this context, the term, “spacer”, shall be understood to mean implants that can be used temporarily in the scope of the two-step exchange of prostheses in septic revision surgeries.

Carrier materials for local antibiotics therapy can be provided as spheres or sphere-like bodies or as bean-shaped bodies. Besides, it is also feasible to produce rod-shaped or disc-shaped carrier materials that contain bone cement made from the paste according to the invention and/or the kit according to the invention. Moreover, the carrier materials can also be threaded onto absorbable or non-absorbable suture material in a bead-like manner.

The uses according to the invention of bone cement described above are known from the literature and have been described therein on numerous occasions.

A contribution to meeting the objects specified above is also made by the use of a filling agent that is poorly soluble or insoluble in a monomer for radical polymerisation, whereby the filling agent is a particulate cross-linked polymethacrylate that can be produced, preferably was produced, through polymerisation of methacrylic acid esters, whereby

i) at least 15% by weight of the methacrylic acid esters used for polymerisation are multifunctional methacrylic acid esters, relative to the total weight of the methacrylic acid esters used in the polymerisation, and

ii) at least 90% by weight of the particles of the filling agent, relative to the total weight of the filling agent, have a particle size of no more than 74 μm,

as component in a composition that contains the monomer for radical polymerisation as polymerisable component and can be cured through polymerisation, preferably in a bone cement paste that can be cured through polymerisation.

The components that were described above in the context of the paste according to the invention as preferred monomer for radical polymerisation and particulate cross-linked polymethacrylate are preferred in the present context as well as monomer for radical polymerisation and as particulate cross-linked polymethacrylate.

The invention shall be illustrated through the examples described in the following, though without limiting the scope of the invention.

### EXEMPLARY EMBODIMENTS

### Example (According to the Scope of the Invention)

Pastes A and B were produced according to example 1 of DE 10 2010 024 653 A1. However, a filling agent produced from a mixture of 80% by weight methylmethacrylate and 20% by weight ethylene glycol dimethacrylate through suspension polymerisation using a thermally disintegrating radical initiator was used as insoluble polymethylmethacrylate in pastes A and B. The grain size of the filling agent was less than 63 μm.

Pastes A and B were stored at room temperature over night and then mixed with each other at a weight ratio of 1:1. This immediately resulted in pastes that were tack-free and cured after a few minutes.

### Reference Example (Not According to the Scope of the Invention)

Pastes A and B having the same composition as in the preceding example were prepared except that polymethylmethacrylates with a lesser degree of cross-linking were used as particulate filling agent. A cross-linked polymer was synthesised from a mixture of 5% by weight ethylene glycol dimethacrylate and 95% by weight methylmethacrylate and a second polymer was synthesised from a mixture of 10% by weight ethylene glycol dimethacrylate and 90% by weight methylmethacrylate. The pastes produced with said particulate polymers after storage at room temperature over night were extremely viscous, leather-like, and it was not possible to mix them to form a tack-free cement dough.

1. A paste comprising at least one polymer for radical polymerisation, at least one polymer that is soluble in said at least one monomer for radical polymerisation, and at least one filling agent that is poorly soluble or insoluble in said at least one monomer for radical polymerisation, whereby the filling agent is a particulate cross-linked polymethacrylate that was produced through polymerisation of methacrylic acid esters, whereby
i) at least 15% by weight of the methacrylic acid esters used for polymerisation are multi-functional methacrylic acid esters, relative to the total weight of the methacrylic acid esters used in the polymerisation, and
ii) at least 90% by weight of the particles of the filling agent, relative to the total weight of the filling agent, have a particle size of no more than 74 μm.

2. The paste according to claim 1, wherein at least 20% by weight of the methacrylic acid esters used for polymerisation are multifunctional methacrylic acid esters, relative to the total weight of the methacrylic acid esters used in the polymerisation.

3. The paste according to claim 1, wherein the paste contains two different particulate cross-linked polymethacrylates, P1 and P2, that implement features i) and ii), wherein
P1 can be obtained through polymerisation of 90 to 100% by weight multifunctional methacrylic acid esters and 0 to 10% by weight monofunctional methacrylic acid esters, each relative to the total weight of the methacrylic acid esters used in the polymerisation, and
P2 can be obtained through polymerisation of 20 to 40% by weight multifunctional methacrylic acid esters and 60 to 80% by weight monofunctional methacrylic acid esters, each relative to the total weight of the methacrylic acid esters used in the polymerisation.

4. The paste according to claim 1 wherein the multifunctional methacrylic acid ester is selected from the group consisting of ethylene glycol dimethacrylate, butan-1,4-diol-dimethacrylate, hexan-1,6-diol-dimethacrylate, dodecan-1,12-diol-dimethacrylate, diethylene glycol dimethacrylate, trimethylene glycol trimethacrylate, glycerol 1,2,3-trimethacrylate, and pentaerythritol tetramethacrylate.

5. The paste according to claim 1 wherein the monomer for radical polymerisation is a methacrylic acid ester.

6. The paste according to claim 1 wherein the paste additionally contains at least one polymerisation initiator, at least one polymerisation accelerator or at least one polymerisation initiator and one polymerisation accelerator.

7. The paste according to claim 1 wherein the soluble polymer is selected from the group consisting of poly(methacrylic acid methylester), poly(methacrylic acid ethylester), poly-(methylmethacrylic acid propylester), poly(methacrylic acid isopropylester), poly(methylmethacrylate-co-methylacrylate), poly(styrene-co-methylmethacrylate), and a mixture of at least two of said polymers.

8. The paste according to claim 1 wherein the paste contains 15 to 85% by weight, relative to the total weight of the paste, of the filling agent that is poorly soluble or insoluble in the at least one monomer for radical polymerisation.

9. A kit comprising a paste A and a paste B,
wherein
(a) paste A comprises
(a1) at least one monomer for radical polymerisation;
(a2) at least one polymer that is soluble in (a1); and
(a3) at least one polymerisation initiator;
(b) paste B comprises contains
(b1) at least one monomer for radical polymerisation;
(b2) at least one polymer that is soluble in (b1); and
(b3) at least one polymerisation accelerator;
and whereby at least one of the pastes A and B contains, as component (a4) and/or (b4), respectively, at least one filling agent that is poorly soluble or insoluble in (a1) and/or (b1), respectively, as defined in any one of the claims 1 to 5.

10. The kit according to claim 9, wherein the monomer (a1) and/or (b1) for radical polymerisation is a methacrylic acid ester.

11. The kit according to claim 9 wherein the polymerisation initiator (a3) is a barbituric acid derivative.

12. The kit according to claim 11, wherein the barbituric acid derivative (a3) is selected from the group consisting of 1-mono-substituted barbiturates, 5-mono-substituted barbiturates, 1,5-di-substituted barbiturates, and 1,3,5-tri-substituted barbiturates.

13. The kit according to claim 9 wherein paste A contains an amount of the at least one polymerisation initiator (a3) in a range of 0.1 to 10% by weight, relative to the total weight of paste A.

14. The kit according to claim 9 wherein the polymerisation accelerator (b3) is at least one heavy metal compound selected from the group consisting of heavy metal salts and heavy metal complexes.

15. The kit according to claim 14, wherein the heavy metal compound is selected from the group consisting of copper(II) hydroxide, basic copper(II) carbonate, and a mixture of at least two thereof

16. The kit according to claim 9 wherein paste B contains an amount of the polymerisation accelerator (b3) in a range of 0.0005 to 0.5% by weight, relative to the total weight of paste B.

17. The kit according to claim 9 wherein paste A and paste B contain an amount of the at least one monomer (a1) and/or (b1) for radical polymerisation in a range of 15 to 85% by weight, each relative to the total weight of paste A and/or paste B, respectively.

18. The kit according to claim 9 wherein the polymer (a3) and/or (b3) that is soluble in (a1) and/or (b1), respectively, is selected from the group consisting of poly(methacrylic acid methylester), poly(methacrylic acid ethylester), poly-(methylmethacrylic acid propylester), poly(methacrylic acid isopropylester), poly(methylmethacrylate-co-methylacrylate), poly(styrene-co-methylmethacrylate), and a mixture of at least two of said polymers.

19. The kit according to claim 9 wherein paste A contains 15 to 85% by weight, relative to the total weight of paste A, of a filling agent (a4) according to claim 1, and paste B contains less than 5% by weight, relative to the total weight of paste B, of a filling agent (b4) according to claim 1.

20. The kit according to claim 19, whereby paste A contains an amount of the polymer (a2) that is soluble in (a1) in a range of 1 to 25% by weight, relative to the total weight of paste A, and paste B contains an amount of the polymer (b2) that is soluble in (b1) in a range of 25 to 85% by weight, relative to the total weight of paste B.

21. The kit according to claim 9 wherein paste A contains 15 to 85% by weight, relative to the total weight of paste A, of a filling agent (a4) that is defined in claims 1 to 5, and paste B contains 15 to 85% by weight, relative to the total weight of paste B, of a filling agent (b4) that is defined in claims 1 to 5.

22. The kit according to claim 21, whereby paste A contains an amount of the polymer (a2) that is soluble in (a1) in a range of 5 to 50% by weight, relative to the total weight of paste A, and/or paste B contains an amount of the polymer (b5) that is soluble in (b1) in a range of 5 to 50% by weight, relative to the total weight of paste B.

23. (canceled)

24. A method for the mechanical fixation of articular endoprostheses, for covering skull defects, for filling bone cavities, for femuroplasty, for vertebroplasty, for kyphoplasty, for the manufacture of spacers, and for the production of carrier materials for local antibiotics therapy comprising applying the paste according to claim 1 to a patient in need thereof.
